(19) United States
(12) Patent Application Publication
(10) Pub. No.: US 2012/0010531 A1
(43) Pub. Date: Jan. 12, 2012

(54) Small-Animal Unit for Muscle Injury, Muscle Testing and Muscle Training in Vivo

(75) Inventors: Robert J. Bloch, Baltimore, MD (US); Joseph A. Roche, Baltimore, MD (US); Richard M. Lovering, Ellicott City, MD (US); Patrick G. De Deyne, Duxbury, MA (US); Wenlong Tang, Catonsville, MD (US); Victor T. Grubbs, Clinton, MD (US); Jeffrey A. Michael, Middle River, MD (US); William T. Sinclair, Towson, MD (US)

(73) Assignee: University of Maryland, Baltimore, Baltimore, MD (US)

(21) Appl. No.: 13/177,255

(22) Filed: Jul. 6, 2011

Related U.S. Application Data
(60) Provisional application No. 61/362,090, filed on Jul. 7, 2010.

(51) Int. Cl. A61B 5/103

(52) U.S. Cl. 600/587

(57) ABSTRACT

The invention provides a system for measuring contractile torque of skeletal muscles, performing muscle training programs, and inducing contraction-induced injury. The system is versatile and precise to measure contractile torque, train muscles, and perform contraction-induced injury protocols on living rodents. The system also allows for repeated studies of the same animal over time, thus resembling longitudinal human studies, minimizing the effect of animal-to-animal variability, and reducing the total number of animals that need to be studied.

### CROSS REFERENCE TO RELATED APPLICATION

This application is based upon and claims benefit of copending and co-owned U.S. Provisional Patent Application Ser. No. 61/362,090 entitled “Small-Animal Unit for Muscle Injury, Muscle Testing and Muscle Training in vivo”, filed with the U.S. Patent and Trademark Office on Jul. 7, 2010 by the inventors herein, the specification of which is incorporated herein by reference.

### GOVERNMENT LICENSE RIGHTS

This invention was made with government support under grant number K01 HD01165 awarded by the National Institutes of Health/National Center for Medical Rehabilitation Research (NIH/NCMRR). The government has certain rights in the invention.

### BACKGROUND

1. Field of the Invention

The present invention relates generally to an apparatus and method for measuring contractile torque of skeletal muscles, performing contraction-induced muscle injury protocols, and performing muscle training protocols, particularly in rodents.

2. Description of the Background

Skeletal muscles make up almost half the weight of the human body. Injuries to muscles result in varying levels of disability. Despite the high prevalence, morbidity, and economic burden associated with muscle injuries, the mechanisms underlying injury and recovery from injury are poorly understood. Understanding these mechanisms would not only help in the management of muscle injuries, but would also improve our understanding of the mechanisms that underlie skeletal muscle diseases, such as muscular dystrophies, in which muscles are more susceptible to injury or are unable to recover from injury as effectively as healthy muscle. Animal models of injury and muscle disease are therefore very useful in understanding the mechanisms underlying injury and recovery. Over the years, various types of equipment have been developed to induce injury in muscles and quantitatively measure their strength. However, most of these models are limited in their ability to perform in vivo measurements of muscle strength and study the contributions of the arc of motion to injury.

### SUMMARY

Accordingly, it is an object of the present invention to provide a device for testing and measuring contractile torque of skeletal muscles that avoids the disadvantages of the prior art.

It is an object of the present invention to provide a device to injure the muscle controlling the movement of the foot by single or repetitive lengthening contractions over a broad range of arcs of motion and velocities.

It is another object of the present invention to provide a device for testing and measuring torque produced by the group of muscles at the ankle joint.

It is another object of the present invention to provide a custom designed, light weight footplate. A related object of the present invention to provide a torque cell attached to the footplate. A further related object is to provide a motor to move the footplate.

Another object of the present invention is to provide an electrical stimulator to stimulate the nerve that innervates a group of muscles.

Another object of the present invention is to provide a stabilization device that stabilizes the limb under test.

A further object of the invention is to provide software to synchronize torque readings, electrical stimulation, and movement of the footplate.

These and other objects of the present invention are accomplished by providing a system for measuring contractile torque of skeletal muscles, performing muscle training programs, and inducing contraction-induced injury. The system is versatile and precise to measure contractile torque, train muscles, and perform contraction-induced injury protocols on living rodents. The system also allows for repeated studies of the same animal over time, thus resembling longitudinal human studies, minimizing the invasiveness of the procedures and the effects of animal-to-animal variability, as well as reducing the total number of animals that need to be studied.

Broadly, the system can be used to test neuromuscular function, train muscles, and perform contraction-induced injury protocols on muscles. At a basic science level, the system can be used to study normal and pathological physiology of the neuromuscular system. At an applied science level, the system is useful for pre-clinical testing of the effects of potential physical, pharmacological, cell-based, and gene therapies on the neuromuscular system.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a general overview of the system according to the present invention.

FIG. 2 shows a testing stand according to an embodiment of the present invention.

FIG. 3 shows another view of the testing apparatus according to an embodiment of the present invention.

FIG. 4 shows the testing stand in use according to an embodiment of the present invention.

FIG. 5 is a schematic of a power supply/control unit according to an embodiment of the present invention.

FIG. 6 shows a block diagram for a virtual instrument according to an embodiment of the present invention.

FIG. 7 shows a graphical user interface for a virtual instrument according to an embodiment of the present invention.

### DETAILED DESCRIPTION OF EXEMPLARY EMBODIMENTS

The invention summarized above and defined by the enumerated claims may be better understood by referring to the following description, which should be read in conjunction with the accompanying drawings. This description of an embodiment, set out below to enable one to build and use an implementation of the invention, is not intended to limit the invention, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

The Small-animal Unit for Muscle Injury, Muscle Testing and Muscle Training in vivo, abbreviated and referred to hereafter as the “SUMITT”, is a device for measuring contractile torque of skeletal muscles, performing muscle training programs, and inducing contraction-induced injury with rodents. The basic design of the SUMITT makes it the most versatile and precise system to measure contractile torque, train muscles, and perform contraction-induced injury protocols on living rodents. The SUMITT also allows for repeated studies of the same animal over time, thus resembling longitudinal human studies, minimizing the effect of animal-to-animal variability, and reducing the total number of animals that need to be studied.

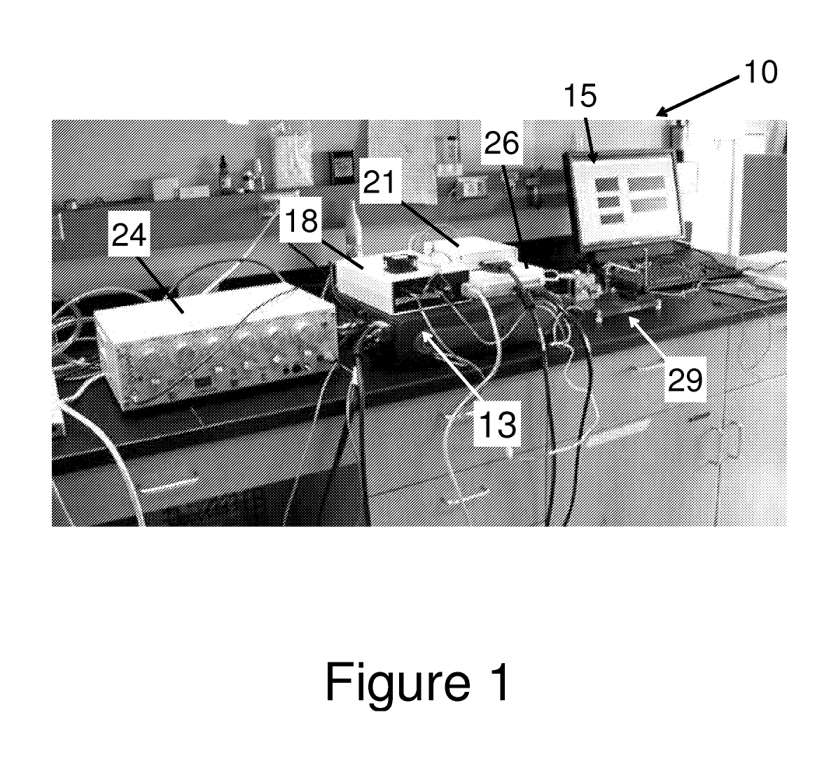

As illustrated in FIG. 1, the SUMITT, referred to broadly at 10, comprises the following components or groups of components. A computer 13 having a monitor 15 provides a graphic user interface for the SUMITT 10. The computer 13 includes custom written software that controls coordination and operation of the SUMITT 10. In some embodiments, the SUMITT 10 includes various components as further described herein. A control unit 18 connects the computer 13 with a stepper motor controller 26 through a universal motion interface 21. A specially designed test stand 29 holds the test subject on a platform 47 for muscle testing, as described below. An electrical stimulator 24 provides stimulation to the selected nerve for the muscle(s) to be tested.

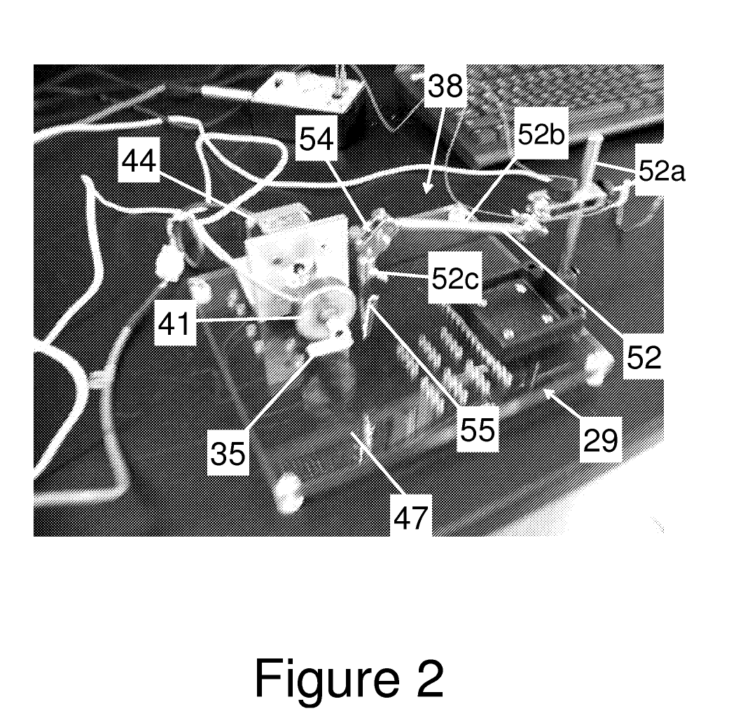

Referring to FIGS. 2-4, the test stand 29 includes the platform 47 for holding the test animal, a limb stabilization device 38, a torque cell 41, and a stepper motor 44. The torque cell 41 and stepper motor 44 are attached to a lightweight footplate 35. The stepper motor 44 is used to move the footplate 35, and the torque cell 41 is used to measure torque produced by the group of dorsiflexor muscles at the ankle joint.

A test animal is held on the platform 47 by a limb stabilization device 38 that stabilizes the tibia or femur of the animal. In some embodiments, an additional instrument to control inhalation of an anesthetic, such as isoflurane, may be provided to maintain general anesthesia when the animal is on the SUMITT 10. The electrical stimulator 24 uses one or more electrodes 50 to stimulate the nerve that innervates the group of dorsiflexor muscles.

The SUMITT 10 also includes hardware, such as the universal motion interface 21, to convert analog to digital signals, and hardware, such as the control unit 18 and computer 13, to synchronize torque readings, electrical stimulation, and movement of the footplate 35. The computer 13 includes a processor having custom written software that is used to synchronize torque readings, electrical stimulation, and movement of the footplate 35.

In one embodiment, the torque cell 41 may be a QWFK-8M miniature reaction torque transducer from Honeywell Sensotec having a transducer range of approximately 20 in-oz to 300 in-lb, with nearly infinite sensitivity. The footplate 35 is a custom-built footplate design and weighs approximately 1.0 gm. The footplate 35 is fabricated from a single block of aluminum or other lightweight metal and has a sleeve that goes over the shaft of the torque cell 41, to which it is held firmly in place by a small screw. The surface of the footplate 35 on which the animal's foot is placed includes a plurality of holes approximately 1 mm in diameter punched out at regular intervals in order to reduce the weight of the footplate 35 without significantly compromising tensile strength. The design ensures that most of the torque applied by the ankle muscles of an animal under test is transferred to the torque cell 41 and stepper motor 44, and is influenced minimally by the weight of the footplate 35.

In one embodiment, the stepper motor 44 may be a bidirectional stepper motor from Portescap-Danaher Motion LLC (4.1 VDC 1.8DEG BI), which requires lower power consumption and provides superior microstepping torque linearity. The stepper motor 44 provides full 360° movement capability. This range is important for performing injury protocols that require a large degree of stretching to be superimposed on the muscle contractions.

Limb stabilization is achieved with a limb stabilization assembly 38. The stabilization assembly 38 allows adjustment in X, Y, and Z planes with spherical pivoting points that facilitate optimal placement of the foot on the footplate 35. The stabilization assembly 38 includes an adjustable arm 52 and a transosseus stabilization pin 57. The arm 52 is made of several sections 52a, 52b, 52c, and can be adjusted at a plurality of linkage joints 54 to enable adjustments in the superior-inferior, medio-lateral, and anterior-posterior planes of movement. The variable adjustment capability makes it possible to adjust for variability in the placement of one or more stabilization pins 57. At the end of the adjustable arm 52 is a clamp 55, such as an “alligator clip”. When an animal is studied on the SUMITT 10, a stabilization pin 57 of suitable gauge is passed through the head of the tibia or the femur (typically 27 G for mice, 18 G for rats). After the stabilization pin 57 is passed through the bone, the clamp 55 is positioned to hold the ends of the stabilization pin 57. The gap between the clamp 55 and the limb under test allows greater access to the stimulation point for the nerve that innervates the desired muscles. While less invasive stabilization methods can be used, they tend to obscure the stimulation points and provide insufficient stability to the leg, thereby compromising the results.

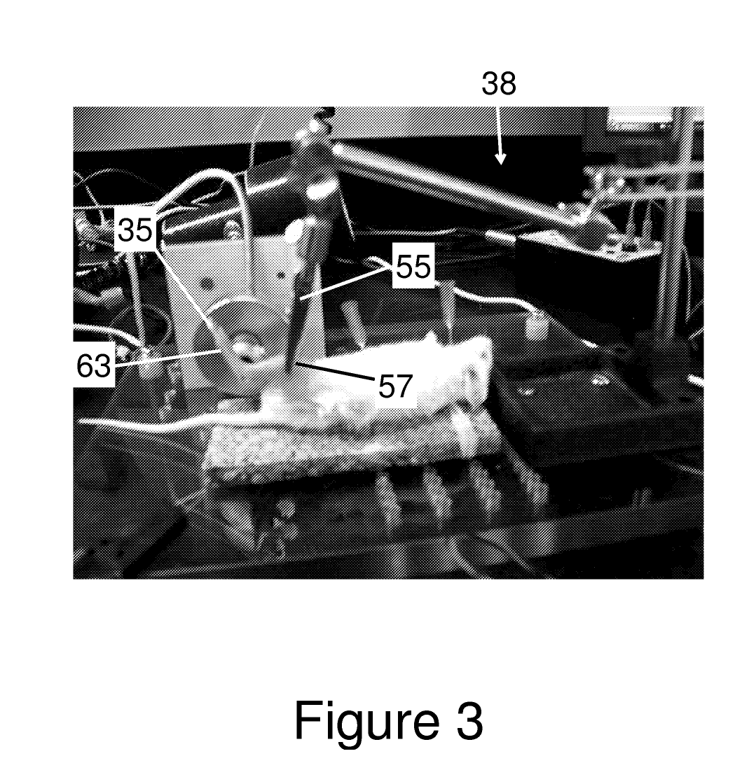

Some variability in placement of the stabilization pin 57 is inevitable when working with mice and other small animals; even small differences in stabilization pin placement affects torque readings. The arm 52 of the stabilization assembly 38 can be adjusted to align the tibia and place the animal's foot on the footplate 35, where it is stabilized. In some embodiments, adhesive tape 63 can be used to hold the animal's foot on the footplate 35, as shown in FIG. 3. This configuration enables performing highly reproducible measurements, training protocols, and contraction-induced injury protocols. Added precision is achieved by the ability to compensate for variability in the placement of the stabilizing pin 57 by adjusting the arm 52 of the stabilizing assembly 38.

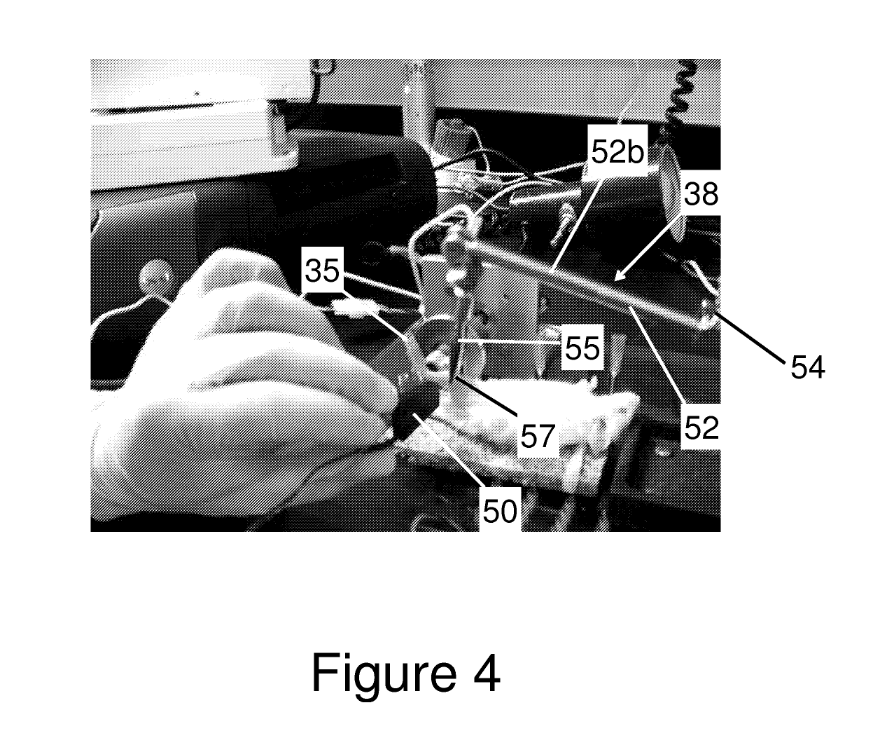

In one embodiment, the electrical stimulator 24 may be an S48 square pulse stimulator from Grass Instruments. The electrical stimulator 24 can be triggered by an external source, such as by the computer 13, which is useful in timing the stimulation with torque recording and movement of the footplate 35. The electric current amplitude, pulse duration, pulse frequency, and pulse-train duration can be customized. In one embodiment, a PSIU6 stimulation isolation unit from Grass Instruments can be used to limit the maximum current. Referring to FIG. 4, electrically elicited muscle contractions are obtained by stimulating an appropriate nerve, which innervates the selected muscles. Different types of electrodes 50 can be used depending on specific requirements of each experiment. However, the most commonly used electrode 50, which is least invasive, is a bipolar electrode, such as a Simple Electrode 506824 from Harvard Apparatus. Typically, the electrode 50 is placed on the skin, close to the head of the fibula where the fibular nerve is superficial.

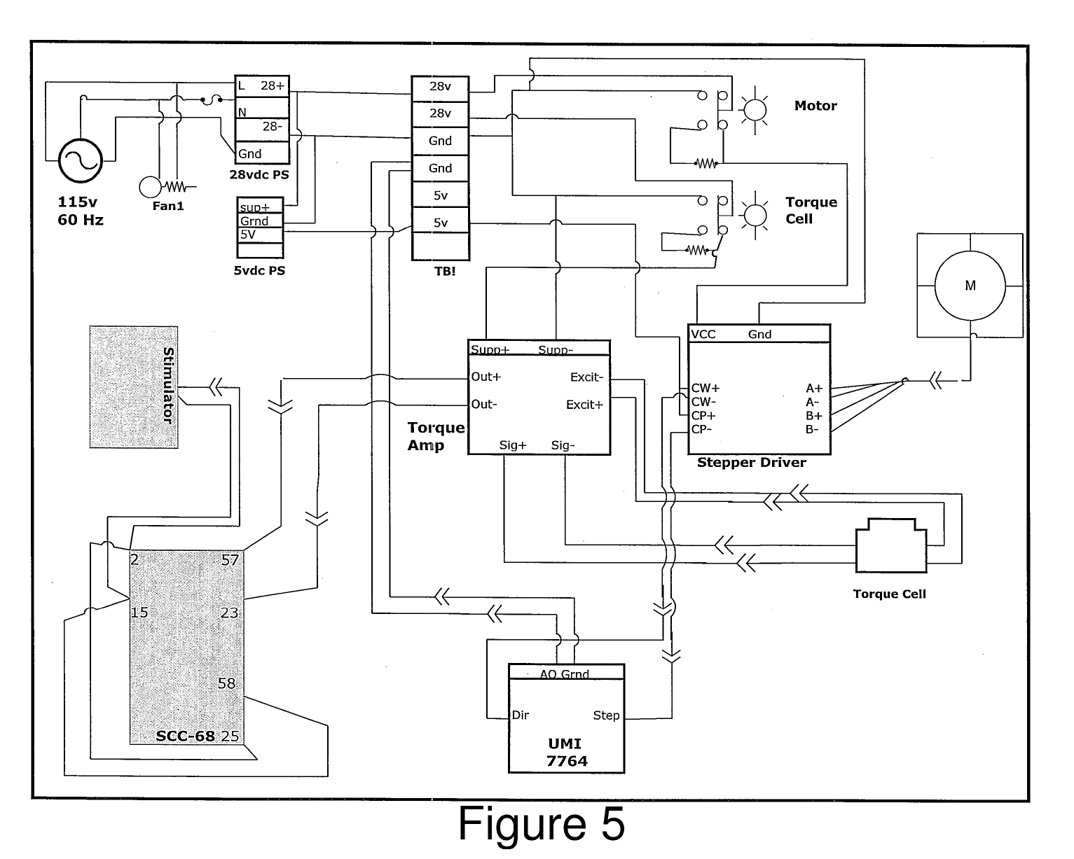

The stepper motor 44 can be controlled by a controller card such as one provided by National Instruments, model PCI-7330. An analog-to-digital converter (NI PC I-6220, M Series DAQ with NI-DAQmx driver software) and connecting modules 21 (UMI-7764, 20 Mhz Encoders, 4 Axis Mot Wiring Connectivity Module; and SCC-68 I/O Connector with 4 SCC Module Slots) enable the conversion of analog to digital signals, and the incorporation of hardware to synchronize torque readings, electrical stimulation, and movement of the footplate 35. A schematic of a power supply/control unit is shown in FIG. 5.

For the animal under test, general anesthesia may be induced and maintained by a commercial tabletop anesthesia device, such as one available from VetEquip™. Isoflurane may be used as an anesthetic. In some embodiments, the animal may be kept warm by an incandescent light bulb, such as a 60 W bulb placed approximately 15 cm away from the animal. Other external sources to keep the animal warm may be used, such as thermal gel packs or electrical heating pads. A more sophisticated warming system that controls the temperature of the platform 47 on which the animal rests may be incorporated into the SUMITT 10. For example, a system that enables warming of the surface by running warm water through channels of tubing under the platform 47 may be used.

Custom-written, original software is used to synchronize torque readings, electrical stimulation, and movement of the footplate 35. In some embodiments, the software for the SUMITT is used to collect isometric torque and eccentric torque data. The software also controls the movement of the footplate 35, and the timing of onset of electrical stimulation. The software can be written on a platform such as LabVIEW™, which allows creation of virtual instruments (VI) that can perform measurement on the test animal. Virtual instruments can be written to automate injury and fatigue protocols and perform measurements on concentric contractions. The versatility of the software platform enables the possibility to create additional VIs to increase the functionality of the SUMITT 10.

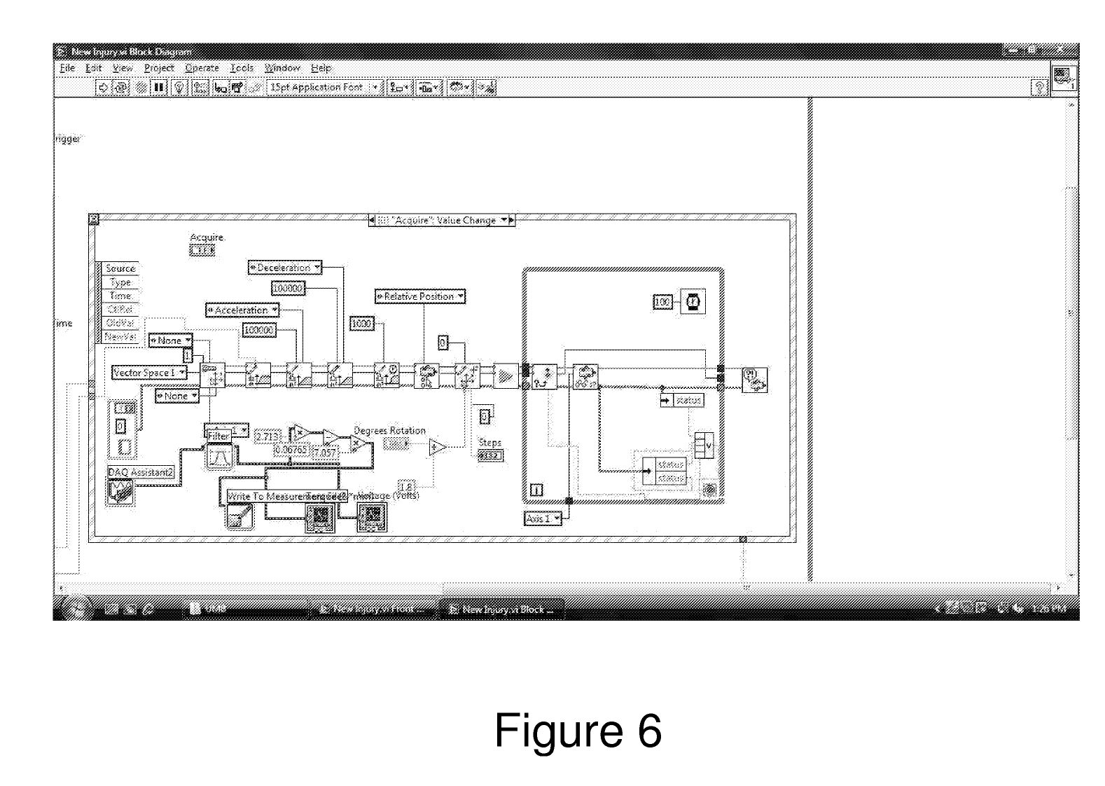

FIG. 6 shows a block diagram of a VI according to one embodiment of the present invention. The event structure illustrated in FIG. 6 is for controlling movement of the footplate 35 and triggering of the electrical stimulator 24. The functions illustrated in the lower left of the diagram are for saving torque data and for displaying the torque signals.

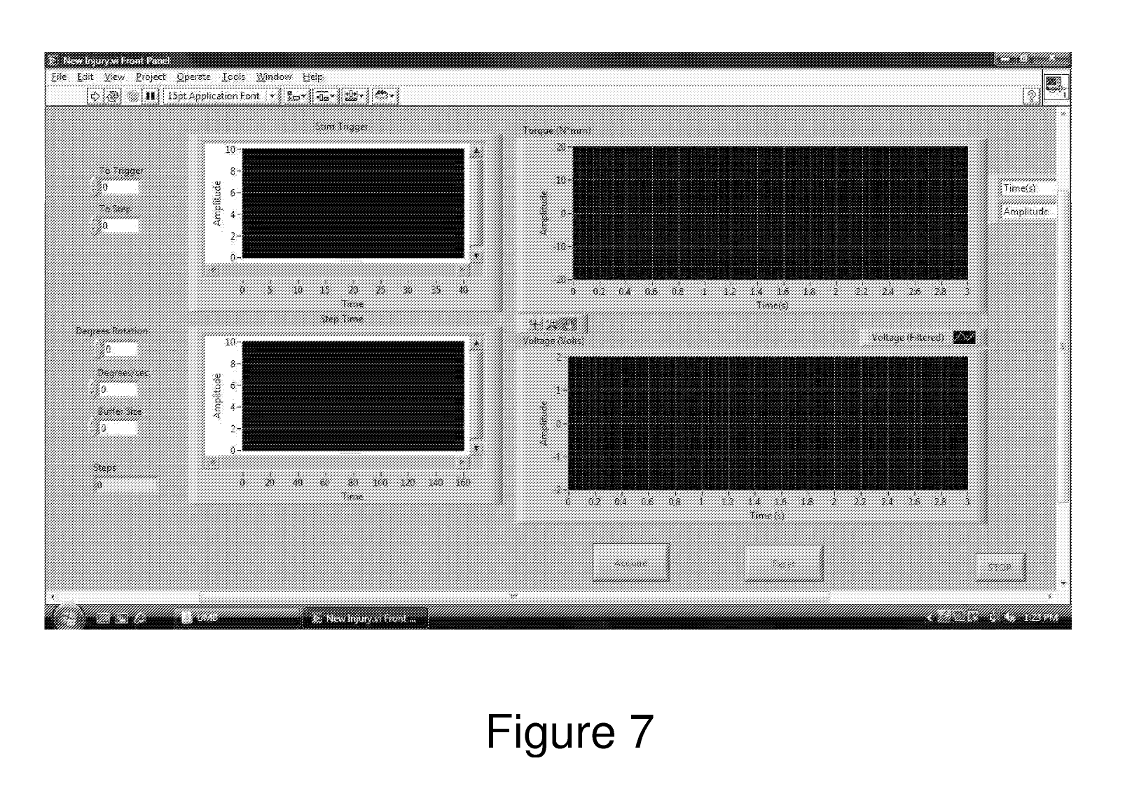

In one embodiment, the virtual instrument (VI) for performing torque measurements and contraction-induced injury protocols can generate several waveforms on graphs, such as shown in FIG. 7. On the top left of FIG. 7 is the “Stimulation Trigger”, which can display the signal that triggers the electrical stimulator 24. On the bottom left is the “Step Time”, which can show the timing of the movement of the footplate 35. On the top right, a display of torque in Newton-millimeters can be shown and on the bottom right, a display of torque in volts can be shown. A user can accurately correlate the timing of the stimulation and the resultant movement. A value is entered by a user in the boxes labeled “To Trigger”, “To Step”, “Degrees Rotation”, and “Degrees/Sec”, which determine the timing of triggering the electrical stimulator 24, the delay between onset of stimulation and movement of the footplate 35, the angle through which the footplate 35 moves, and the angular velocity at which the footplate 35 moves. The software calculates values for “Buffer Size” and “Step” based on the user-entered values. The commands written in the VI are executed by clicking the “Acquire” button. Data obtained as described above are displayed in real time. The “Reset” button restores the footplate 35 to its starting position and the “Stop” button terminates execution of the VI.

To use the SUMITT 10, an animal is prepared per institutional guidelines for sterile procedures. Hair is removed from the appropriate limb using a commercial depilatory cream such as Magic™ Softsheen Carson. A pin 57, of appropriate gauge, is then passed through the head of the tibia or femur and stabilized by the stabilization assembly 38 using the clamp 55. The animal's foot is secured onto the footplate 35 by appropriate means. In some embodiments, adhesive lab tape 63 may be used to secure the animal's foot. Optimal placement of the electrode 50 is assessed by using single pulses to obtain maximum twitch contractions of the stimulated muscles. The custom-written software is used to set the position of the footplate 35 and to synchronize initiation of electrical stimulation with movement of the footplate 35. The software also controls angular velocity of the footplate movement and the degrees of rotation through which the footplate should move. The software also enables recording of torque produced during isometric, eccentric, and concentric contractions of the ankle dorsiflexors. With appropriate software algorithms, the SUMITT can perform measurements and control training regimens and contraction-induced injury protocols on the ankle dorsiflexors and plantarflexors.

The design of the SUMITT 10 is very simple. The platform 47 on which the test animal rests during protocols can be made as small as 15 cm×20 cm. This is enabled by the fact that the torque cell 41, stepper motor 44, and limb stabilization assembly 38 are separate components. This enables the SUMITT 10 to be used in conjunction with intravital microscopy. Imaging of a muscle in vivo, during injury, is important to understanding the dynamic changes that take place in muscle cells during muscle injury.

The SUMITT is very versatile and can be compared favorably with commercial isokinetic machines, such as those manufactured by Biodex and KinCom that are used routinely for human testing and training. Currently the SUMITT is designed for testing, training, and performing injury protocols on muscles that move the ankle joint in rodents, but the versatility of the SUMITT can be expanded for use with other muscle groups, such as the hamstrings, quadriceps femoris, and triceps brachii, which are commonly studied in experiments involving rodents. Additionally, the SUMITT can be scaled to fit other animals for studies of rabbits, cats, dogs, etc.

The invention has been described with references to specific embodiments. While particular values, relationships, materials and steps have been set forth for purposes of describing concepts of the invention, it will be appreciated by persons skilled in the art that numerous variations and/or modifications may be made to the invention as shown in the disclosed embodiments without departing from the spirit or scope of the basic concepts and operating principles of the invention as broadly described. It should be recognized that, in the light of the above teachings, those skilled in the art can modify those specifics without departing from the invention taught herein. Having now fully set forth certain embodiments and modifications of the concept underlying the present invention, various other embodiments as well as potential variations and modifications of the embodiments shown and described herein will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention might be practiced otherwise than as specifically set forth herein. Consequently, the present embodiments are to be considered in all respects as illustrative and not restrictive.

What is claimed is:

1. A system for muscle injury, training, and torque measurement in vivo, comprising:
a torque cell attached to a footplate; and
a stepper motor attached to the footplate;
a stabilization assembly for stabilizing a limb for testing; and
an electrical stimulator to innervate one or more muscles;
wherein movement of the footplate and electrical stimulation is digitally synchronized using software.

2. The system according to claim 1, further comprising a stepper motor controller.

3. The system according to claim 1, further comprising at least one converter to convert output from the torque cell to a digital signal.

4. The system according to claim 1, said stabilization assembly further comprising an adjustable arm and transosseus stabilization pin.

5. The system according to claim 4, said adjustable arm further comprising a clamp at one end of the arm.

6. The system according to claim 5 wherein said clamp is releasably attached to an end of the stabilization pin.

7. The system according to claim 4 wherein said adjustable arm can be adjusted at at least three different points.

8. The system according to claim 7 wherein said adjustable arm can be adjusted in the superior-inferior, medio-lateral, and anterior-posterior planes of movement.

9. The system according to claim 1 wherein said stepper motor comprises a bidirectional stepper motor.

10. The system according to claim 1 wherein said stepper motor provides 360° movement.

11. The system according to claim 1, further comprising a computer having a monitor.

12. The system according to claim 11, said computer further comprising software for use in a computer processor adapted to execute said software.

13. The system according to claim 12 wherein said software controls coordination and operation of the system.

14. The system according to claim 13 wherein said software is used to synchronize torque readings from said torque cell with electrical stimulation from said electrical stimulator and movement of the footplate.

15. The system according to claim 1, further comprising a test stand for holding an animal for testing.

16. The system according to claim 15, said test stand comprising a platform and the stabilization assembly.

17. The system according to claim 15, said test stand further comprising a device for keeping the animal under test warm.

18. The system according to claim 1, further comprising an anesthesia system for an animal for testing.

19. The system according to claim 1, said electrical stimulator further comprising at least one electrode.

20. The system according to claim 1 wherein said electrical stimulator can be triggered by an external source.
